(19) United States
(12) Patent Application Publication
(10) Pub. No.: US 2022/0380422 A1
(43) Pub. Date: Dec. 1, 2022

(54) METHOD FOR TREATING MUSCLE ATROPHY AND/OR OBESITY AND COMPOSITION FOR USE IN SAID METHOD

(71) Applicant: Lirsamax, Inc., Newton, MA (US)

(72) Inventor: Samudra S. Gangopadhyay, Newton, MA (US)

(21) Appl. No.: 17/825,659

(22) Filed: May 26, 2022

Related U.S. Application Data
(60) Provisional application No. 63/194,601, filed on May 28, 2021.

(51) Int. Cl. C07K 14/47; A61P 35/00; A61P 43/00; A61K 9/00

(52) U.S. Cl. CPC C07K 14/4703; A61P 35/00; A61P 43/00; A61K 9/0019

(57) ABSTRACT

Method for treating muscle atrophy and composition for use in the same. According to one embodiment, the method may be used to treat muscle-wasting conditions like sarcopenia and cancer cachexia and may involve administering to a person suffering from such a condition an effective amount of FST288 and/or one or more variants thereof. In another embodiment, excess fat accumulation and/or obesity may be treated by administering to a person an effective amount of FST288 and/or one or more variants thereof. In yet another embodiment, metabolic activity and/or sprinting activity may be increased by administering to a person an effective amount of FST288 and/or one or more variants thereof. In still yet another embodiment, all types of cancer and/or Alzheimer's disease may be treated by administering to a person suffering from cancer and/or Alzheimer's disease an effective amount of FST288 and/or one or more variants thereof.

### CROSS-REFERENCE TO RELATED APPLICATIONS

The present application claims the benefit under 35 U.S.C. 119(e) of U.S. Provisional Patent Application No. 63/194,601, inventor Samudra S. Gangopadhyay, filed May 28, 2021, the disclosure of which is incorporated herein by reference.

### BACKGROUND OF THE INVENTION

The present invention relates generally to muscle atrophy and relates more particularly to a novel method and composition for use in treating muscle atrophy. In particular, the present invention may be useful as a treatment for muscle-wasting conditions like sarcopenia and cancer cachexia. In addition, the present invention also relates generally to obesity and fat accumulation and is also directed at a method and composition for use in treating obesity and/or reducing fat accumulation. The present invention further relates to a method and composition for use in increasing metabolic activity and/or for use in increasing sprinting activity, as well as for use in curing all types of cancer and/or Alzheimer's disease.

Muscle atrophy is wasting of muscle, as a result of which there is a loss of muscle mass and strength. (See Ding et al., “An Overview of Muscle Atrophy,” Adv Exp Med Biol, 1088:3-19 (2018); Schiaffino et al., “Mechanisms regulating skeletal muscle growth and atrophy,” The FEBS journal, 280(17):4294-4314 (2013); Cohen et al., “Muscle wasting in disease: molecular mechanisms and promising therapies,” Nature reviews. Drug discovery, 14(1):58-74 (2015); Ebert et al., “Skeletal Muscle Atrophy: Discovery of Mechanisms and Potential Therapies,” Physiology (Bethesda, Md.), 34(4):232-239 (2019); and Sartori et al., “Mechanisms of muscle atrophy and hypertrophy: implications in health and disease,” Nature communications, 12(1):330 (2021), all of which are incorporated herein by reference.) Atrophied muscles are smaller than normal muscles and result from a decrease in muscle mass. If a muscle does not get sufficient use, the body will eventually break the muscle down to conserve energy. This phenomenon suggests that lack of physical activity is a reason for muscle loss. While a lack of physical activity accounts for some instances of muscle atrophy, such a lack of physical activity does not account for all instances of muscle atrophy as several other factors can come into play.

For example, poor nutrition can give rise to many health issues including muscle atrophy. While a lack of availability of healthy food is a big problem in underdeveloped countries, many medical conditions, such as, but not limited to, irritable bowel syndrome (IBS), celiac disease, and cancer, also impair the ability of a body to absorb nutrients. With these types of medical conditions, muscle atrophy is a big concern; moreover, muscle atrophy can create other complications as well. Cachexia is a wasting syndrome that causes extreme weight loss and muscle-wasting. A significant loss of appetite or unintentional weight loss, despite consuming a large number of calories, is often a sign of a disease, such as cancer, AIDS, heart failure, or advanced chronic obstructive pulmonary disease (COPD). (See Cretoiu et al., “Nutritional Considerations in Preventing Muscle Atrophy,” Adv Exp Med Biol, 1088:497-528 (2018), which is incorporated herein by reference.)

Muscle atrophy may also be the result of other causes. Neurological problems caused by injury or a health condition can damage the nerves that control muscles, resulting in a condition called neurogenic muscle atrophy. In such a case, muscles stop contracting because they no longer receive signals from the corresponding nerve(s). (See Mary et al., “Neuromuscular diseases: Diagnosis and management,” Orthop Traumatol Surg Res, 104(1S):S89-S95 (2018), which is incorporated herein by reference.)

Some diseases and medical conditions can render a patient impaired of movement. Examples of such diseases and conditions that can contribute to muscle atrophy include the following: amyotrophic lateral sclerosis (ALS), also called Lou Gehrig's disease, multiple sclerosis, arthritis, myositis, and polio. (See Atherton et al., “Control of skeletal muscle atrophy in response to disuse: clinical/preclinical contentions and fallacies of evidence,” Am J Physiol Endocrinol Metab, 311(3):E594-604 (2016), which is incorporated herein by reference.)

Certain genetic disorders that are related to the neuromuscular system can also disable muscle contractile activity and cause progressive weakness and loss of muscle mass. (See Emery et al., “The muscular dystrophies,” Lancet, 359(9307):687-95 (2002), which is incorporated herein by reference.)

Sarcopenia has been defined as an age-related, involuntary loss of skeletal muscle mass and strength. (See Evans, “What is sarcopenia?” J Gerontol A Biol Sci Med Sci, 50 Spec No, 5-8 (1995); and Volpi et al., “Muscle tissue changes with aging,” Curr Opin Clin Nutr Metab Care, 7(4):405-10 (2004), both of which are incorporated herein by reference.) The decline of skeletal muscle mass with age is one of the most important causes of functional decline and loss of independence in older adults. (See Tinetti et al., “Falls, injuries due to falls, and the risk of admission to a nursing home,” N Engl J Med, 337(18):1279-84 (1997), which is incorporated herein by reference.) Age-related neurological decline, inflammatory pathway activation, declines in activity, hormonal changes, chronic illness, fatty infiltration, and poor nutrition are the causes widely considered for loss of muscle mass and loss of muscle function or strength. (See Volpi et al., “Muscle tissue changes with aging,” Curr Opin Clin Nutr Metab Care, 7(4): 405-10 (2004); Holloszy et al., “The biology of aging,” Mayo Clin Proc, 75 Suppl, S3-8; discussion S8-9 (2000); and Vandervoort, “Aging of the human neuromuscular system,” Muscle Nerve, 25(1):17-25 (2002), all of which are incorporated herein by reference.) The U.S. Census Bureau projects that older adults will edge out children in population size by the year 2034, i.e., people age 65 and over are expected to number 77.0 million while children under age 18 will number 76.5 million. Decreased muscle mass has serious consequences at old age as a lack of muscle strength creates problem with mobility, frailty, falls and fractures, decreased activity levels, diabetes, and a loss of physical function and independence.

There is no direct comprehensive estimate of people suffering from muscle-wasting-related diseases; however, one can estimate overall hardship if one considers the following: First, cancer is the second leading cause of death globally and is responsible for an estimated 9.6 million deaths in 2018. Globally, about 1 in 6 deaths is due to cancer (according to the World Health Organization). Second, as many as one in 141 Americans has celiac disease although most do not know it (according to the National Institute of Diabetes and Digestive and Kidney Diseases). Third, studies suggest that about 12 percent of people in the United States have irritable bowel syndrome (according to the National Institute of Diabetes and Digestive and Kidney Diseases). Fourth, around 15% of the global population—over a billion people—lives with some form of disability, of whom 2-4% experience significant difficulties in functioning (according to the World Health Organization).

Obesity is a complicated disease that has more than one cause. (See Apovian, “Obesity: definition, comorbidities, causes, and burden,” Am J Manag Care, 22(7 Suppl):s176-85 (2016); Camacho et al., “Is the calorie concept a real solution to the obesity epidemic?” Glob Health Action, 10(1):1289650 (2017); Singh et al., “Molecular genetics of human obesity: A comprehensive review,” C R Biol, 340(2):87-108 (2017); and Zamboni et al., “Sarcopenia and obesity,” Curr Opin Clin Nutr Metab Care, 22(1):13-19 (2019), all of which are incorporated herein by reference.)

Obesity is not someone's fault, and it is not just about food. Obesity is diagnosed by body mass index (BMI), a measure of weight relative to height. According to the United States Centers for Disease Control and Prevention (CDC), if a BMI is within a range of 25.0 to <30, the BMI is categorized as falling within the overweight range; additionally, if a BMI is 30.0 or higher, the BMI is categorized as falling within the obese range. The CDC also describes that “BMI does not measure body fat directly, but research has shown that BMI is moderately correlated with more direct measures of body fat obtained from skinfold thickness measurements, bioelectrical impedance, underwater weighing, dual-energy x-ray absorptiometry (DXA) and other methods.”

Several factors can bring about an increased BMI. Such factors can include the following: (1) Psychological factors associated with stress and other concerns. (See van der Valk et al., “Stress and Obesity: Are There More Susceptible Individuals?” Curr Obes Rep, 7(2): 193-203 (2018), which is incorporated herein by reference.); (2) An imbalance of calories in and calories burned. This is often attributable to a lack of physical activity from long daily commutes and desk jobs. Not all communities have safe spaces to run, bike, or walk. (See Camacho et al., “Is the calorie concept a real solution to the obesity epidemic?” Glob Health Action, 10(1):1289650 (2017), which is incorporated herein by reference.); (3) Sleep deprivation due to socioeconomic reasons—people who do not get enough sleep may weigh more than people who do. (See Cooper et al., “Sleep deprivation and obesity in adults: a brief narrative review,” BMJ Open Sport Exerc Med, 4(1):e000392 (2018), which is incorporated herein by reference.); (4) Appetite Signals/Hormones signal hunger and fullness of the stomach. Timely eating habits are not maintained always due to involvement/workload at job places. Disruption of appetite signal is a cause of increased body weight; (5) Prescription medications, some of which can cause weight gain of up to several pounds each month. (See Leslie et al., “Weight gain as an adverse effect of some commonly prescribed drugs: a systematic review,” QJM, 100(7):395-404 (2007), which is incorporated herein by reference.); (6) Environmental Factors, such as being surrounded by television ads, billboards, and images that promote the consumption of foods and beverages that are high in calories and fat. Some neighborhoods have little or no access to fresh, healthy foods; (7) Genetic Factors that can impact the likelihood of obesity. There is no cure for such genetic defects, but the deficiencies can be managed by alternative care and by changing habits. The World Health Organization (WHO) reports that, in 2016, 39% of men and 39% of women aged 18+ were overweight (BMI≥25 kg/m2) and 11% of men and 15% of women were obese (BMI≥30 kg/m2). Nearly 2 billion adults worldwide were overweight and, of these, more than half a billion were obese; and (8) The natural process of aging. Aging alters adipose tissue composition and function, resulting in insulin resistance and ectopic lipid storage. Changes in adipose tissue function promote a chronic state of low-grade systemic inflammation. Ultimately, obesity accelerates aging by enhancing inflammation and increasing the risk of age-associated diseases.

In view of the above, there is clearly a need for treatments for addressing muscle atrophy and/or obesity.

### SUMMARY OF THE INVENTION

It is an object of the present invention to provide a novel method for treating muscle atrophy and/or excess fat accumulation and/or aging. In particular, such a method may be useful in treating conditions like sarcopenia and/or cancer cachexia and may also be useful in increasing metabolic activity and/or may be useful in increasing sprinting activity, as well as potentially being useful in curing certain types of cancer and/or Alzheimer's disease.

Therefore, according to one aspect of the invention, there is provided a method of treating sarcopenia or cancer cachexia, the method comprising administering to a person suffering from sarcopenia or cancer cachexia an effective amount of FST288 and/or one or more variants thereof.

In a more detailed feature of the invention, the administering step may comprise administering an effective amount of FST288.

In a more detailed feature of the invention, the administering step may comprise administering an effective amount of one or more variants of FST288.

In a more detailed feature of the invention, the one or more variants of FST288 may comprise one or more peptide sequences that differ from FST288 by an insertion, deletion and/or substitution of one or more peptides while still retaining some efficacy in treating sarcopenia or cancer cachexia.

In a more detailed feature of the invention, the one or more variants of FST288 may comprise at least one of (i) FST288 with a first tag attached; and (ii) a peptide sequence with a second tag attached, and the peptide sequence may differ from FST288 by an insertion, deletion and/or substitution of one or more peptides while still retaining some efficacy in treating sarcopenia or cancer cachexia.

In a more detailed feature of the invention, the first tag and the second tag may be the same kind of tag.

In a more detailed feature of the invention, the first tag and the second tag may be different kinds of tags.

In a more detailed feature of the invention, each of the first tag and the second tag may be selected from the group consisting of a His-tag, a GST tag, and a FLAG-tag.

In a more detailed feature of the invention, the FST288 and/or the one or more variants may be dissolved in a solvent of phosphate-buffered saline containing 20% glycerol.

In a more detailed feature of the invention, the FST288 and/or the one or more variants thereof may be present in the solvent at a concentration of about 5 mg of the FST288 and/or the one or more variants per kg of body weight of the person dissolved in about 100 μl of the solvent.

In a more detailed feature of the invention, said administering step may comprise administering to the person one or more doses, and each dose may constitute about 100 μl of a solution comprising the FST288 and/or the one or more variants dissolved in the solvent.

In a more detailed feature of the invention, said administering step may comprise administering to the person one dose per day for a period of at least 2-3 days.

In a more detailed feature of the invention, said administering step may comprise administering to the person one dose per day for a period of at least 2-3 weeks.

In a more detailed feature of the invention, said administering step may comprise administering by at least one of subcutaneous administration, intramuscular administration, intravenous administration, and intrathecal administration.

In a more detailed feature of the invention, said administering step may comprise administering by subcutaneous administration.

In a more detailed feature of the invention, said administering step may comprise administering to the person one or more doses, and each dose may contain FST288 and/or the one or more variants thereof at any amount.

According to another aspect of the invention, there is provided a method of preventing/reversing muscle loss or reducing fat accumulation, the method comprising administering to a person an effective amount of FST288 and/or one or more variants thereof.

In a more detailed feature of the invention, said administering step may comprise administering to the person one or more doses, and each dose may contain FST288 and/or the one or more variants thereof at any amount.

The present invention is also directed at a composition for use in performing the aforementioned method.

Additional objects, as well as features and advantages, of the present invention will be set forth in part in the description which follows, and in part will be obvious from the description or may be learned by practice of the invention. In the description, reference is made to the accompanying drawings which form a part thereof. Embodiments of the invention will be described in sufficient detail to enable those skilled in the art to practice the invention, and it is to be understood that other embodiments may be utilized and that changes may be made without departing from the scope of the invention. The following detailed description is, therefore, not to be taken in a limiting sense, and the scope of the present invention is best defined by the appended claims.

### BRIEF DESCRIPTION OF THE DRAWING

The accompanying drawing, which is hereby incorporated into and constitutes a part of this specification, illustrates certain aspects of the invention and, together with the description, serves to explain the principles of the invention. In the drawings wherein like reference numerals represent like parts:

FIG. 1 is a graph depicting cell viability (in vitro assay) in the presence of FST288 as discussed in the Example.

### DETAILED DESCRIPTION OF THE INVENTION

The present invention is directed, at least in part, at a method for treating muscle-wasting conditions, such as, but not limited, to sarcopenia and cancer cachexia. Sarcopenia is an age-related, involuntary loss of skeletal muscle mass and strength that is typically accompanied with an increased accumulation of visceral fat. Cancer cachexia is a complex metabolic syndrome associated with most types of cancer and is characterized by a dramatic loss of skeletal muscle mass and body weight.

In addition, the present invention is also directed, at least in part, at a method for treating excess fat accumulation, particularly obesity.

Additionally, the present invention is also directed, at least in part, at a method for increasing metabolic activity and/or for use in increasing sprinting activity.

Moreover, the present invention is also directed, at least in part, at a method for curing all types of cancer and/or Alzheimer's disease.

According to one aspect of the invention, a method for treating a muscle-wasting condition and/or excess fat accumulation may comprise administering to a person suffering from the muscle-wasting condition and/or excess fat accumulation an effective amount of Follistatin288 (FST288) and/or one or more variants thereof. In fact, in one embodiment, any kind of muscle loss (atrophy) may be treated by administering to a patient suffering from muscle loss (atrophy) an effective amount of FST288 and/or one or more variants thereof.

According to another aspect of the invention, a method for increasing metabolic activity and/or sprinting activity may comprise administering to a person an effective amount of FST288 and/or one or more variants thereof.

According to still another aspect of the invention, a method for curing some or all types of cancer and/or Alzheimer's disease may comprise administering to a person suffering from cancer and/or Alzheimer's disease an effective amount of FST288 and/or one or more variants thereof.

FST288 is a recombinant peptide, and its peptide sequence is as follows:

GNCWLRQAKNGRCQVLYKTELSKEECCSTGRLSTSWTEEDVNDNTLFKW
MIFNGGAPNCIPCKETCENVDCGPGKKCRMNKKNKPRCVCAPDCSNITW
KGPVCGLDGKTYRNECALLKARCKEQPELEVQYQGRCKKTCRDVFCPGS
STCVVDQTNNAYCVTCNRICPEPASSEQYLCGNDGVTYSSACHLRKATC
LLGRSIGLAYEGKCIKAKSCEDIQCTGGKKCLWDFKVGRGRCSLCDELC
PDSKSDEPVCASDNATYASECAMKEAACSSGVLLEVKHSGSCN

Variants of FST288 may include, but are not limited to, peptide sequences that differ from FST288 by the insertion, deletion and/or substitution of one or more peptides, provided that the variant peptide sequence retains at least some efficacy for its stated purpose.

Variants of FST288 may also include peptide sequences of the aforementioned types to which one or more identical or different tags may be attached, examples of such tags including, but not being limited to, His-tags (i.e., polyhistidine-tags), GST tags (i.e., glutathione S-transferase tags), and FLAG-tags (i.e., DYKDDDDK tags).

FST288, as well as its variants, can be expressed in eukaryotic and/or prokaryotic expression systems and should be manufactured toxin-free. As an example, FST288 and/or its variants may be expressed in E. coli as a recombinant peptide. The purity and toxicity of the expressed peptide should be appropriate for use as a biologic (e.g., purity >90%; endotoxin <0.1 EU/mg). Proper folding of the protein is important. An exemplary method for expressing and purifying human FST288 is set forth below.

Follistatin288 (FST288, amino acid residue no. 30-317) cDNA may be cloned into the Sal1/Xho1 site of pET30 and expressed with a His-tag in E. coli BL21(DE3) by induction with IPTG. The cells may then be resuspended in buffer containing 100 mM Tris-HCl, pH 8.0, and 10 mM NaCl and sonicated on ice. The insoluble portion of the lysed cell suspension may then be separated by centrifugation, and the pellet may be solubilized with a buffer containing 50 mMTris-HCl, pH 8.0, 8 M urea and 100 mM PMSF. His-tagged FST288 may be purified with a HisPur cobalt spin column (Thermo scientific, Rockford, Ill.) according to the manufacturer's instructions or using other purification method/column/procedure etc. The purified protein, in elution buffer containing 8 M urea, may be diluted (1:4) with 200 mM Tris-HCl, pH 10.0, and 2 mM DTT and incubated on ice for 4-5 hours. The diluted protein may be dialyzed against Tris buffer (10 mM Tris-HCl, pH 8.0, and 1 mM NaCl) or PBS at 4° C., with several changes. The purified protein may be subsequently passed through Detoxi-Gel (Endotoxin Removal Gel), Thermo Scientific, to remove bacterial endotoxins and stored at −80° C. with 15-20% glycerol. (Expression of the peptide, FST288, utilizing other eukaryotic/prokaryotic/insect/viral systems may be possible).

For administration to a patient, FST288 and/or its variants may be dissolved in a suitable solvent. For example, FST288 and/or its variants may be dissolved in PBS (phosphate-buffered saline) containing 20% glycerol. The FST288 may be present in the solvent at a suitable concentration, such as, but not limited to, about 5 mg FST288 per kg of patient body weight. A dose may have a suitable volume, such as, but not limited to, about 100 μl, and doses may be administered on a regular basis, such as one dose per day for a period of time ranging from up to a few days to several weeks or longer. The concentration and/or dose may be varied, depending on the condition being treated.

The solution containing FST288 and/or its variants may be administered to a person in any suitable manner, with injection (i.e., parenteral administration) being preferred. More specifically, administration by injection may include one or more of the following routes: subcutaneous (i.e., under the skin); intramuscular (i.e., in a muscle); intravenous (i.e., in a vein); and intrathecal (i.e., around the spinal cord). Subcutaneous injection may be a preferred injection route.

Without wishing to be limited to any particular theory behind the invention, it is believed that the invention may work as follows: Myostatin, which is a protein, is a member of the transforming growth family beta (TGF-β) superfamily. Myostatin is a negative regulator of muscle growth. FST288 may be used to block myostatin function and, in so doing, may stimulate the growth of muscle mass.

FST288 may function in one or more of the following ways: (a) to increase body lean mass and muscle mass in a dose-dependent manner; (b) to concomitantly decrease body fat mass along with an increase in muscle growth; and (c) to significantly increase ambulatory activity (measurement of physical activity, and movement).

The following example is given for illustrative purposes only and is not meant to be a limitation on the invention described herein or on the claims appended hereto.

### Example: In Vitro Toxicity of FST288

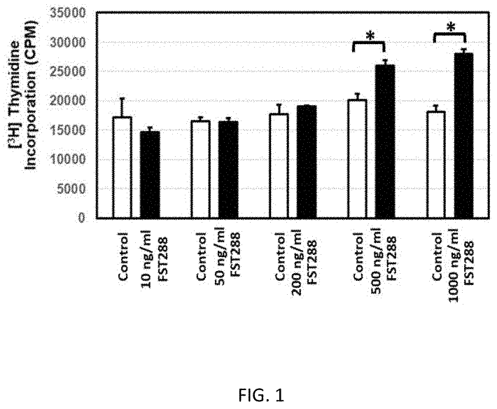

Cell viability was tested to evaluate the toxicity of FST288 on a mouse plasmacytoma cell line, MPC11. The growing of cells in the presence of FST288 was assessed by tritiated thymidine incorporation (see Gangopadhyay, “Systemic administration of Follistatin288 increases muscle mass and reduces fat accumulation in mice,” Sci Rep. 3:2441 (2013), doi:10.1038/srep02441; and Phillips et al., “A sensitive and specific in vitro bioassay for activin using a mouse plasmacytoma cell line, MPC-11,” J Endocrinol., 162(1):111-6 (1999), doi:10.1677/joe.0.1620111, both of which are incorporated herein by reference). As can be seen in FIG. 1, concentrations of 10, 50, and 200 ng/ml FST288 did not change the cellular viability of MPC11 compared to the control (the symbol “*” in FIG. 1 indicates p<0.05). However, a significant increase in tritiated thymidine incorporation was observed at a higher concentration of FST288 (500 ng/ml and above). This data suggests that FST288 at a concentration of 500 ng/ml and above may be a growth stimulator whereas, below 500 ng/ml, FST288 may not have a comparable proliferative effect. In any event, the more significant observation is that FST288 does not cause a toxic effect in vitro as a decrease in cellular viability was not observed. Therefore, it should be safe to use FST288 clinically.

Additional comments regarding the invention are set forth below.

FST288 may be used as a biologic drug for one or more of the following purposes: (1) to stimulate muscle mass growth, (2) to reduce fat accumulation, and (3) to increase endurance and ambulatory activity (measurement of physical activity, and movement). Thus, administration of FST288 may be used to cause a change in body composition, with an increased lean mass/fat mass ratio and improved metabolic parameters for sprinting ability.

In addition to being useful for treating conditions like sarcopenia and cancer cachexia, FST288 may also be useful in preventing and reversing muscle loss, as well as reducing fat accumulation associated with various other conditions or factors (e.g., obesity, lack of physical activity, poor nutrition, neurological problems, medical conditions, genetic disorders, etc.). Depending on the dose and mode of application of the biologic, the drug response may vary.

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A method of treating sarcopenia or cancer cachexia comprising administering to a person suffering from sarcopenia or cancer cachexia an effective amount of FST288 and/or one or more variants thereof.

2. The method as claimed in claim 1 wherein the administering step comprises administering an effective amount of FST288.

3. The method as claimed in claim 1 wherein the administering step comprises administering an effective amount of one or more variants of FST288.

4. The method as claimed in claim 3 wherein the one or more variants of FST288 comprise one or more peptide sequences that differ from FST288 by an insertion, deletion and/or substitution of one or more peptides while still retaining some efficacy in treating sarcopenia or cancer cachexia.

5. The method as claimed in claim 3 wherein the one or more variants of FST288 comprise at least one of (i) FST288 with a first tag attached; and (ii) a peptide sequence with a second tag attached, the peptide sequence differing from FST288 by an insertion, deletion and/or substitution of one or more peptides while still retaining some efficacy in treating sarcopenia or cancer cachexia.

6. The method as claimed in claim 5 wherein the first tag and the second tag are the same kind of tag.

7. The method as claimed in claim 5 wherein the first tag and the second tag are different kinds of tags.

8. The method as claimed in claim 5 wherein each of the first tag and the second tag is selected from the group consisting of a His-tag, a GST tag, and a FLAG-tag.

9. The method as claimed in claim 1 wherein the FST288 and/or the one or more variants thereof are dissolved in a solvent of phosphate-buffered saline containing 20% glycerol.

10. The method as claimed in claim 9 wherein the FST288 and/or the one or more variants thereof are present in the solvent at a concentration of about 5 mg of the FST288 and/or the one or more variants per kg of body weight of the person dissolved in about 100 μl of the solvent.

11. The method as claimed in claim 10 wherein said administering step comprises administering to the person one or more doses, each dose constituting about 100 μl of a solution comprising the FST288 and/or the one or more variants dissolved in the solvent.

12. The method as claimed in claim 11 wherein said administering step comprises administering to the person one dose per day for a period of at least 2-3 days.

13. The method as claimed in claim 12 wherein said administering step comprises administering to the person one dose per day for a period of at least 2-3 weeks.

14. The method as claimed in claim 1 wherein said administering step comprises administering by at least one of subcutaneous administration, intramuscular administration, intravenous administration, and intrathecal administration.

15. The method as claimed in claim 14 wherein said administering step comprises administering by subcutaneous administration.

16. The method as claimed in claim 1 wherein said administering step comprises administering to the person one or more doses, each dose containing FST288 and/or the one or more variants thereof at any amount.

17. A method of preventing/reversing muscle loss or reducing fat accumulation comprising administering to a person an effective amount of FST288 and/or one or more variants thereof.

18. The method as claimed in claim 17 wherein said administering step comprises administering to the person one or more doses, each dose containing FST288 and/or the one or more variants thereof at any amount.
